(19) United States
(12) Patent Application Publication
(10) Pub. No.: US 2023/0157621 A1
(43) Pub. Date: May 25, 2023

(54) METHOD AND APPARATUS FOR PROCESSING SIGNALS FOR DETECTING AND SIGNALLING AN IMMINENT LOSS OF BALANCE OF A SUBJECT AND ASSOCIATED SYSTEM FOR PREVENTIVE DETECTION OF A FALL

(71) Applicant: POLITECNICO DI BARI, BARI (IT)

(72) Inventors: DANIELA DE VENUTO, BARI (IT); GIOVANNI MEZZINA, BARI (IT); MICHELE RUTA, BARI (IT); EUGENIO DISCIASCIO, BARI (IT)

(21) Appl. No.: 17/919,056

(22) PCT Filed: Apr. 12, 2021
(86) PCT No.: PCT/IB2021/052997

§ 371 Date: Oct. 14, 2022

(30) Foreign Application Priority Data
Apr. 16, 2020 (IT) 102020000008059

(51) Int. Cl. A61B 5/00; A61B 5/397; A61B 5/369; A61B 5/11

(52) U.S. Cl. CPC A61B 5/4023; A61B 5/397; A61B 5/369; A61B 5/1117; A61B 5/6801; A61B 5/7282

(57) ABSTRACT

A method for processing physiological signals (S_{EMG}; S_{EEG}) acquired from a subject (S) allows the detection of an imminent loss of balance of the subject and the generation of a signal (Aout) indicating the imminent loss of balance. The method comprises: the reception of a plurality of electromyographic signals (S_{EMG}) representative of a detected muscle activity of a plurality of selected muscles of the subject, as well as a plurality of brain signals (S_{EEG}) acquired by means of an electroencephalogram and representative of a cortical activity of the subject during said muscle activity; the analysis and processing of the electromyographic signals (S_{EMG}) in order to extract a muscle activity pattern, MAP, and generate an indicator of normality/abnormality of the detected muscle activity pattern; the analysis and processing of the brain signals (S_{EEG}) in order to generate one or more cortical response indicators of the subject upon occurrence of said detected muscle activity (I_{EG}g; LF(k)); and a classification step, wherein at least one indicator (MA(k)) of normality/abnormality of the MAP and one or more of said cortical response indicators are correlated to generate a signal (Aout) indicating an imminent loss of balance.

The present invention relates to a method and an apparatus for processing signals acquired from a subject in order to detect an imminent loss of balance of the subject and generate a signal indicating the imminent loss of balance, as well as a system for preventive detection of a fall on the part of the subject. The annual report of the World Health Organization (WHO) on the most common causes of accidents and deaths identifies falls as being the second main cause of deaths due to accidental and non-intentional injury throughout the world [World Health Organization. “WHO global report on falls prevention in older age.” Online Updated (Jan. 16, 2018)]. With reference to data updated to 16 Jan. 2018, the WHO estimates that overall, each year, 646,000 persons die as a result of falls. In the same context, Center for Disease Control (CDC) in the United States estimated that every 19 minutes one adult (65+) dies following a fall, while every 19 seconds an emergency recovery is recorded for the same reason. During 2017 alone, about 37.3 million falls considered to be serious, but not lethal, for which continuous medical assistance was required, were recorded. The report identifies elderly people as being most affected by fall events.

In fact, it is estimated that about 28-35% of persons aged over 65 fall at least once each year. With the advance in the natural ageing process, such situations may increase in number to up to 5-7 events per year.

The WHO identifies the consequences of falls as “devastating”. The physical lesions caused by falls, such as fractures of the hip and the thigh-bone, subdural hematomas, haemorrhages, etc., are often associated with a high mortality and morbidity among elderly persons.

A further and recurring problem associated with the fall event is the probability of elderly persons developing a “fear of repeated falling” following an event associated with the first fall. This fear of a psychological nature results in the loss of mobility and independence of the subjects who experience such an event, resulting in an increasingly more sedentary lifestyle, thus increasing the risk of falling.

It is known that postural control is the result of a complex sequence of movements intended to preserve the state of static or dynamic equilibrium which has become inherent in human beings following their evolution to bipedalism.

Information from different types of sensorial receptors allow the motor system to generate automatic compensatory (feed-back) measures or anticipatory (feed-forward) measures, if the postural response is stressed by an event disturbing the equilibrium (e.g. uneven environments, sudden slipping, tripping, etc.).

Recent statistics support the clinical evidence whereby the natural ageing process alters the natural capacity of the human body to deal with unexpected disturbances in the equilibrium by means of compensatory or anticipatory counter-measures.

Moreover, the illnesses associated with age (which often affect the cerebral circuits) and traumatic events (e.g. serious injuries or loss of the lower limbs) may further aggravate the inability to maintain the correct posture, resulting in a dramatic increase both in the risk of loss of balance and in the seriousness of the falling incidents related thereto.

In this connection, the field of fall detection arises with the primary aim of creating systems or devices able to detect, automatically and with due accuracy, a fall event.

It is known in the sector of fall prevention that most fall events in real life occur as a result of unexpected disturbances which cause a loss of balance of the subject and are characterized by an involuntary nature.

In connection with fall detection strategies, the techniques of Post-fall Mobility Detection (PFMD) which have the aim of detecting the fall event when it has already occurred and evaluating the subsequent state of mobility of the subject being monitored are known. The paradigm forming the basis of these systems envisages that the detection of the fall event should give rise to prompt medical assistance, thus avoiding deaths due to the subject remaining in the lying position for a long period of time.

Despite the increasing technological progress in the sector of PFMD architecture, this type of detection of falls has an intrinsic limitation. Falls may be detected only following impacts, for which reason it is not possible to prevent injuries directly caused by fall impacts.

MEZZINA GIOVANNI ET AL: “EEG/EMG based Architecture for the Early Detection of Slip-induced Lack of Balance”, 2019 IEEE 8TH INTERNATIONAL WORKSHOP ON ADVANCES IN SENSORS AND INTERFACES (IWASI), describes a preliminary study in the sector of early recognition of loss of balance while walking at a constant speed.

In this context, a subject walks at a constant speed on a mechanical treadmill and forced slipping of the subject is specifically induced by means of operation of the mechanical treadmill by an operator.

A multi-sensor architecture acquires physiological electromyography (EMG) signals on the lower limbs and electroencephalography (EEG) signals on the scalp.

With the aim of analysing the muscle activity and the cortical activity during the disturbance induced by the treadmill during constant-speed walking, the EMG and EEG data are analysed a posteriori offline and correlated with the control signal generated by the mechanical treadmill which induced the slipping action.

In greater detail, the EMG data acquired is statistically processed and used to identify anomalous muscle activities and the EEG signals acquired are processed in order to evaluate the simultaneous cortical activity, quantifying a rate of variation in the density of the EEG power spectrum so as to describe the cortical reactivity in five bands of interest.

Thereafter, a network of logical conditions allows the system to recognize in the previously acquired data the slipping induced by the mechanical treadmill, considering the muscular parameters, the cortical response parameters and the mechanical treadmill control signal.

The offline post-processing described in the document is intended exclusively for an a posteriori evaluation of slipping artificially induced by means of a mechanical treadmill controlled by an operator during walking at a constant speed of a harnessed subject on the treadmill.

The system cannot be used to detect sufficiently in advance an imminent involuntary loss of balance (fall) which occurs in daily activity nor to generate a suitable signal indicating the imminent loss of balance.

The technical problem which is posed, therefore, is that of developing pre-impact fall detection systems and strategies which can be used to detect in advance losses of balance of a subject, which may result in a fall, during normal ordinary activity.

The pre-impact fall detection strategies PIFD relate in general to techniques which are able to recognize the fall event before the body hits with force the ground (kinematic situation known as “body-ground impact).

In connection with this problem it is required in particular to provide a method and an apparatus able to detect and/or signal an imminent loss of balance of a subject, for example when the (static or dynamic) equilibrium is unexpectedly disturbed by unexpected events of varying nature.

In this context, it is particularly preferable that the method and the apparatus should be efficient from the point of view of the detection and/or signalling time, allowing in particular the implementation of compensatory action for avoiding falls and/or of actions able to reduce the degree of the body-ground impact, conventionally estimated as being within the range of 700 ms-1000 ms following the event disturbing the equilibrium of the subject. In connection with this problem it is required in particular that the method and the apparatus should be accurate, making it possible in particular to distinguish a loss of balance (or actually fall) from all those movements considered to be ordinary (e.g. walking running, jumping, etc.).

A particular object of the present invention is therefore that of providing an apparatus and a method for detecting and signalling an imminent loss of balance, characterized by a satisfactory degree of accuracy and/or reliability in terms of distinguishing losses of balance of an involuntary nature from the activities of ordinary life, preferably within a detection/signalling time frame suitable for the implementation of compensatory or preventive action.

A further object of the present invention is to provide a pre-impact fall detection system which is simple to implement, in particular being wearable and/or not requiring complicated wiring, so that it can be used by subjects in their daily life.

In connection with this problem it is also preferable that this system should have small dimensions and be easy and inexpensive to produce and assemble.

These results are obtained according to the present invention by means of a method of processing physiological signals according to claim 1 and a processing apparatus according to claim 20.

With a processing method and apparatus according to the invention it is advantageously possible to detect and signal with precision an imminent involuntary loss of balance and therefore a probably imminent fall, owing to cross-analysis and processing of electromyographic signals of the subject, allowing analysis of the muscular activity performed, and of brain signals, making it possible to take into account the cortical involvement of the subject during a reactive response. Using at least one indicator of normality/abnormality of the muscle activity pattern detected on selected muscles of the subject, at least one indicator of normality/abnormality of the generalized cortical response of the subject upon occurrence of said muscle activity, and an indicator of lateralization of the cortical response, which indicates in particular a normality/abnormality of the involvement of the left and right cortical sides in the cortical response, the classification in accordance with the method and the apparatus of the present invention is able to generate a signal indicating an imminent loss of balance in the case of simultaneous presence of anomalies in a generalized cortical response in one or more macro-areas, a non-lateralized anomalous cortical response and a simultaneous abnormality of the muscle activity pattern of the selected muscles.

Therefore, with the method and the apparatus according to the invention it is possible detect in a reliable and rapid manner imminent effectively involuntary losses of balance, also during the normal ordinary activity of the subject. Further preferred embodiments are described in the dependent claims which are fully cited herein.

The present invention relates furthermore to a system for the preventive detection of a fall according to claim 35.

Further details and technical advantages may be obtained from the following description of non-limiting examples of embodiment of the subject of the present invention, provided with reference to the accompanying drawings, in which:

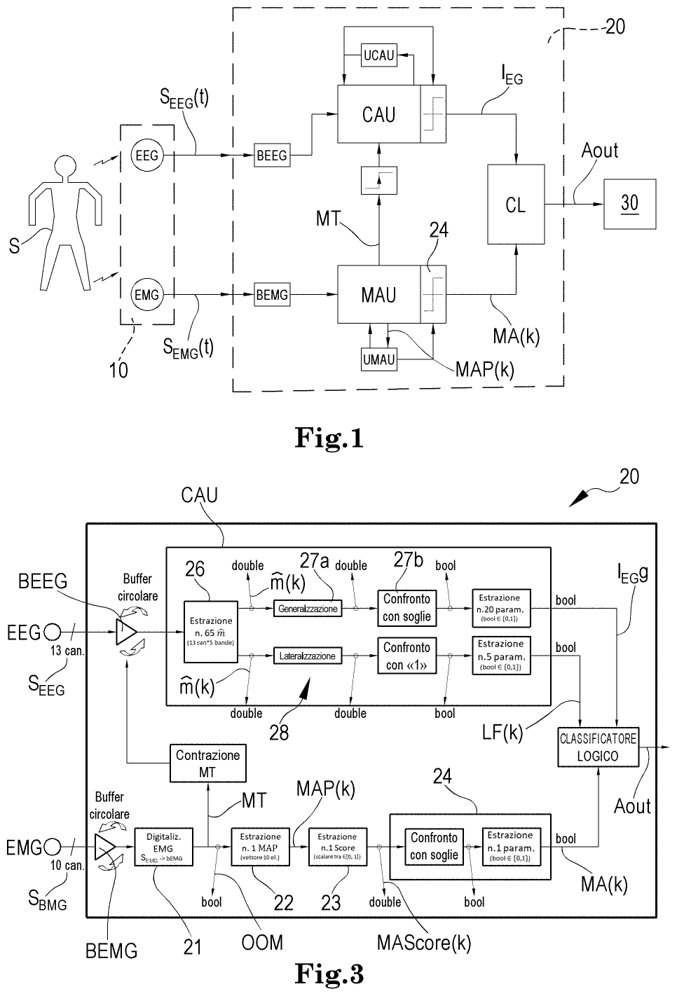

FIG. 1: shows a synthetic and simplified block diagram of an example of a structure of a fall prevention system according to the present invention;

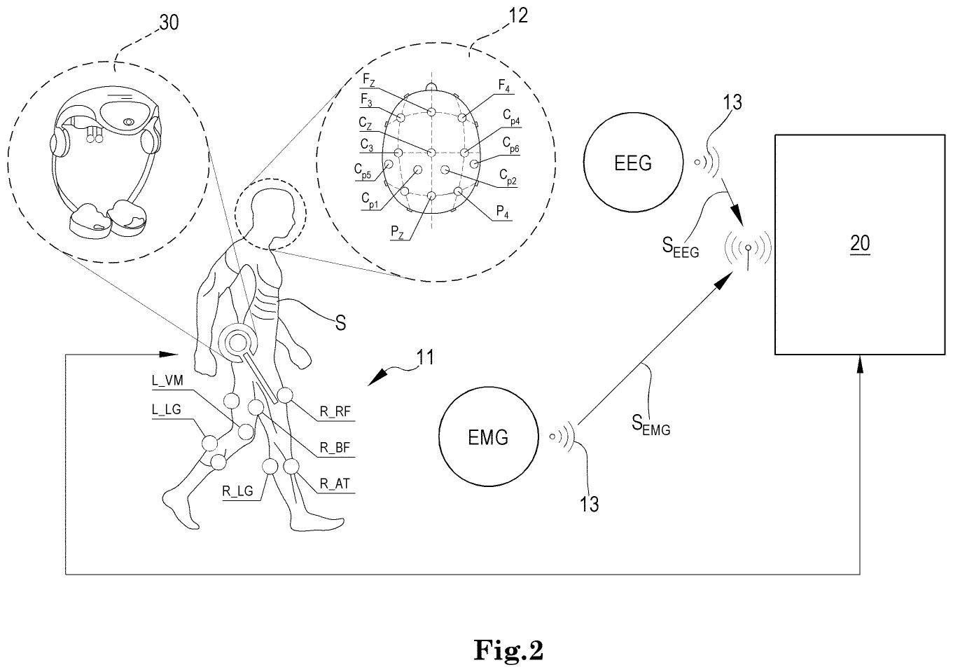

FIG. 2: shows a view, in greater detail, of an example of embodiment of an acquisition unit of the system according to FIG. 1 connected to a processing apparatus of the present invention in turn connected to a unit for implementing a corrective or preventive action;

FIG. 3: shows a schematic diagram of some of the method steps performed by a preferred example of embodiment of a processing apparatus according to the present invention;

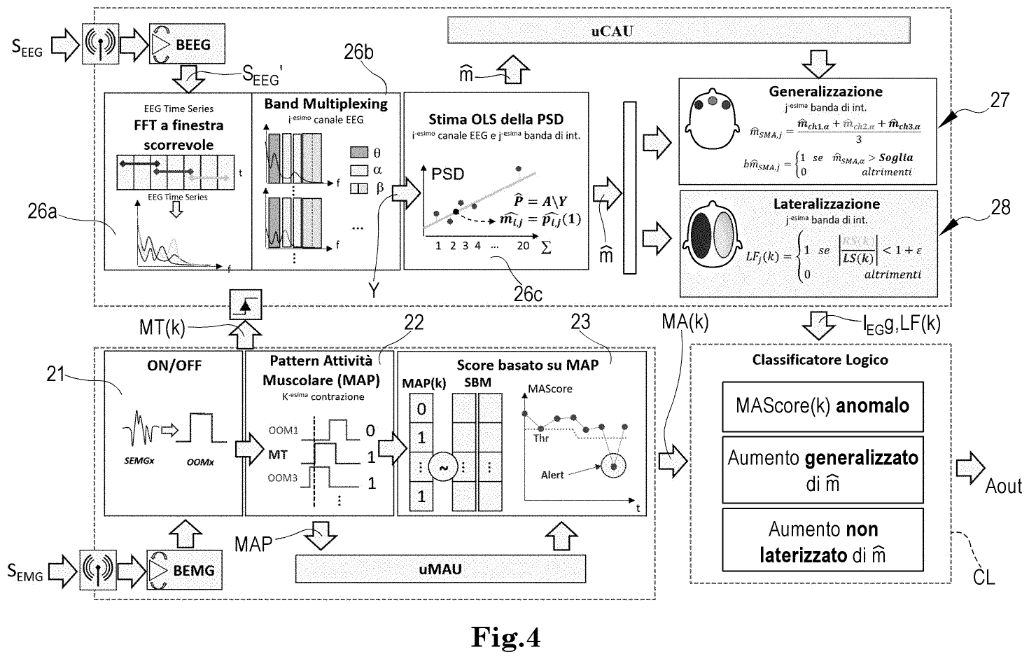

FIG. 4: shows a block diagram illustrating a variation of an example of embodiment of a processing apparatus according to the present invention and the associated method steps implemented by it;

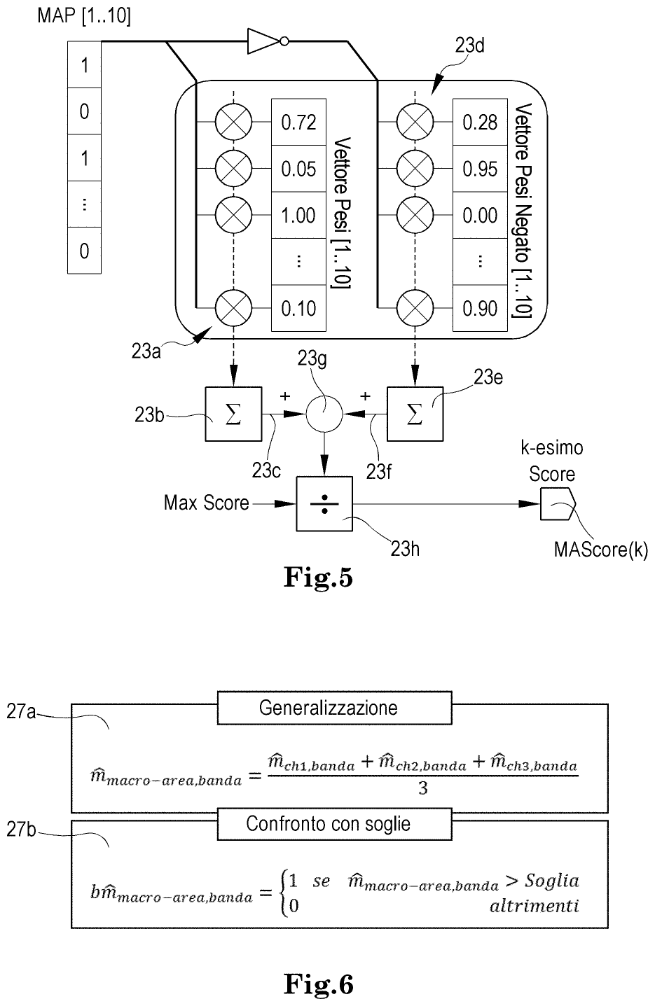

FIG. 5: shows a detailed view of an example of a score calculation block able to quantify the normality/abnormality of the MAP;

FIG. 6: shows a schematic view of a preferred generalization routine performed by a cortical analysis unit;

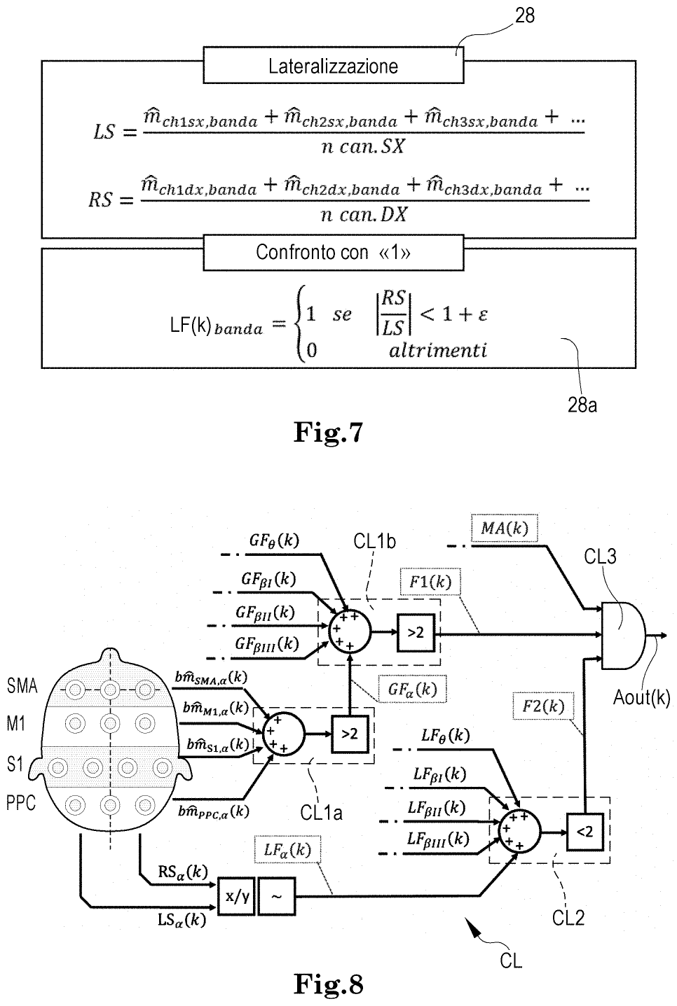

FIG. 7: shows a schematic view of a preferred lateralization routine performed by a cortical analysis unit;

FIG. 8: shows a simplified diagram of a preferred embodiment of a classifier of a processing apparatus according to the present invention.

For the purposes of simplification of the present description the terms “signals”, “data”, “data flows” and “digital flows” will be used substantially as synonyms, where not otherwise specified, it being within the competence of the technical expert to implement the appropriate processing and/or conversion techniques at different points within the processing flow.

Moreover, the terms “indicators” or “parameters” will be used in an equivalent manner, these being understood as referring to a data or an associated data flow or a signal, which conveys given functional information.

Furthermore, below reference will be made to a direction of flow of the data from upstream to downstream, with reference respectively to an upstream part for acquisition/reception of incoming signals input into the system of the invention and downstream part for emission of signals output from the system.

General Structure

With reference to FIG. 1, the figure shows in schematic form a subject S and the general structure of an example of embodiment of the system according to the invention for the preventive detection of a fall of a subject, which comprises:
an acquisition unit 10 for acquiring physiological signals, configured to acquire: a plurality of electromyography (EMG) signals S_{EMG}, acquired at a plurality of selected muscles of the subject and representative of a detected muscle activity of said muscles of the subject, and a plurality of brain signals S_{EEG}, acquired by means of electroencephalogram (EEG) and representative of motor cortical activity of the subject during said muscle activity;
an apparatus 20 for processing physiological signals, designed to receive at its input (by means of suitable connection means and/or interface circuits) the electromyographic signals S_{EMG} and the brain signals S_{EEG} acquired and transmitted by the acquisition unit 10 and to process them so as to generate at least one indicator signal Aout which signals an imminent loss of balance of the subject S;
optionally, a unit 30 for implementing a corrective and/or protective action, configured to receive said indicator signal Aout signalling an imminent loss of balance and to implement at least one corrective and/or preventive action, for example an action designed to restore the equilibrium and prevent falling of the subject and/or an action designed to limit the negative effects of the imminent fall. The unit 30 may be for example an electromechanical unit, for example a robotic unit which can be worn by the subject S.

With reference still to FIG. 1, the processing apparatus 20 according to the present invention comprises preferably a plurality of buffers BEMG for receiving and storing the plurality of electromyographic signals S_{EMG}acquired and transmitted by the acquisition unit 10, and a plurality of buffers BEEG for receiving and storing the plurality of brain signals S_{EEG} acquired and transmitted by the acquisition unit 10.

The buffers BEMG, BEEG may in particular be in the form of circular registers for the continuous and synchronized acquisition of the signals S_{EMG}and S_{EEG}, describing, respectively, the correlated muscle and motor cortical activity of the subject S.

A muscle analysis unit MAU is arranged downstream of the buffers BEMG and configured to analyse and process the plurality of signals S_{EMG}received in order to extract a pattern (profile) of said detected muscle activity (MAP). The muscle analysis unit MAU is also configured to generate at least one indicator (MA(k)) of normality/abnormality of the detected muscle activity pattern, in particular by correlating said extracted muscle activity pattern with a standard muscle behaviour pattern.

A cortical analysis unit CAU is arranged downstream of the buffers BEEG and is configured to process the plurality of signals S_{EEG}received in order to generate a plurality of cortical response indicators I_{EG} indicating a cortical response of the subject upon occurrence of the muscle activity detected by the electromyographic signals acquired by the acquisition unit 10, described in greater detail further below.

According to a preferred aspect and as shown in FIG. 1, the analysis and processing of the brain signals S_{EEG}is started by a trigger signal MT generated in response to a contraction of a reference muscle detected by the unit MAU during the analysis and processing of one or more of said signals S_{EMG}.

A classifier CL is configured to receive at its input the at least one indicator (MA(k)) of normality/abnormality of the detected muscle activity pattern and said cortical response indicators IEG and to process them, correlating them, in order to generate an indicator signal Aout which indicates that an anomaly indicating an imminent loss of balance of the subject has been detected.

### Acquisition Unit

With reference to FIG. 2, the acquisition unit may be a generic multi-sensor interface able to collect, in a combined (synchronized) manner, the data from a plurality of EMG sensors 11 (for example electrodes) arranged, bilaterally, for monitoring the muscle activity of a plurality of selected muscles and from an EEG acquisition device 12 for monitoring a plurality of cortical channels, in particular forming part of the motor area, the supplementary motor area and/or the motor sensor area, in order to acquire electroencephalographic brain signals S_{EEG}of the subject, representative of the motor cortical activity of the subject during said muscle activity,

On the cortical side of the acquisition unit 10, a preferred embodiment envisages sensors designed to monitor at least 13 specific channels selected from among those forming part of the motor, supplementary motor and sensory-motor area. In greater detail, the preferred channels to be monitored are: F3, Fz, F4, C3, Cz, C4, Cp5, Cp1 Cp2, Cp6, P3, Pz, P4. The annotation is provided in accordance with the international positioning system 10-20 of sensors for acquiring electroencephalographic signals S_{EEG}.

Preferably, the electrode O2 (occipital cortical area) is used for noise suppression, the electrode AFz as a ground electrode and the electrode A2 (right ear lobe) as a reference electrode.

According to a recommended acquisition profile the EEG data is sampled at 500 Hz with a 24-bit resolution.

On the muscle side of the acquisition unit 10, according to a preferred embodiment, sensors are provided for monitoring the muscle activity, bilaterally, from the following muscle groups: Anterior Tibial (AT), Lateral Gastrocnemius (LG), Vastus Medialis (VM), Rectus Femoris (RF) and Biceps Femoris (BF). According to a recommended acquisition profile the EMG signals are sampled at 500 Hz with a resolution of ≥8 bits.

A corresponding number of electromyographic (EMG) signals S_{EMG}is emitted by the acquisition unit 10 and transmitted to the processing unit 20.

In the preferred embodiment, the acquisition unit is provided with transmission means 13, which are preferably wireless.

The acquisition unit 10 is preferably of the type which can be worn so as to allow the subject complete freedom of movement and in general easier use in daily life. Preferred wearability characteristics include a small volume, in particular as regards the physical dimensions of the wearable device, the weight and the weight distribution, such as to be compatible with the normal activity of the subject, and/or low biomechanical effects, in particular a configuration of the sensor devices 11 such that the functional positioning of the sensor nodes does not influence the posture and the musculoskeletal load of the person wearing it. In this connection, the positioning of the node of the sensors must preferably avoid unnatural movements and favour regular movements and undistorted postures.

With reference to the muscle side of the acquisition unit 10, each of the EMG sensor nodes 11 is preferably provided with:
an Analog Digital Converter (ADC) with a resolution of at least 16 bits and a sampling frequency not less than 500 Hz; and/or
an RF interface 13 for wireless communication with the processing unit 20; and/or
a compartment for housing a dedicated battery.

With reference to the cortical side of the acquisition unit 10, it comprises preferably at least 15 electrodes, in particular positioned in the following positions of the 10-20 international system: F3, Fz, F4, C3, Cz, C4, Cp5, Cpl Cp2, Cp6, P3, Pz, P4, Afz and A2. According to a preferred embodiment, the acquisition device EEG comprises:
physical precautions for minimizing the impedance shift artefacts and cable movement. Possible known solutions are auto electrode headphones spaced according to the 10-20 model with flexible PCB connections. Preferable for reading reliability headphones using conductive gels with electrode-skin adaptation; and/or
an Analog Digital Converter (ADC) for each channel, with a resolution of at least 24 bits and a sampling frequency not less than 500 Hz; and/or
an RF interface for wireless communication with the central processing unit; and/or
a dedicated battery.

Unit for Implementing a Corrective and/or Preventive Action

FIG. 2 also show in schematic form an example of a unit 30 for implementing a corrective and/or preventive action in the form of an electromechanical apparatus, in particular an exoskeleton which can be worn by the subject S, configured to receive said signal Aout indicating an imminent loss of balance and to implement a corrective and/or preventive action designed to restore the equilibrium and prevent the subject from falling.

The corrective action may be for example operation of the exoskeleton in order to restore the equilibrium of the subject. A preventive action may be for example the operation of a device for preventing injury from falling, such as an airbag or the like.

Processing Apparatus and Method

With reference to FIG. 3 a preferred example of the signal processing method of the present invention is described in relation to an example of a processing apparatus 20 designed to receive at its input the at least 13 brain signals S_{EEG}and the at least 10 signals S_{EMG} acquired and transmitted by the acquisition unit 10 described above, to process them in order to detect an imminent loss of balance of the subject S and to emit an associated signal Aout indicating the imminent loss of balance.

It will be clear to the person skilled in the art that the processing unit 20 described here as a plurality of functional units, blocks and elements may be implemented by any suitably programmed electronic device, whereby the various functional units, blocks or elements may be in form of hardware, software or firmware or a combination thereof and may be combined or separate units, depending on the choice of the designer or programmer.

As may be deduced, the method comprises the reception on the buffers BEMG of the plurality of electromyographic signals S_{EMG} representing the detected muscle activity of a plurality of selected muscles of the subject; and the reception on the buffers BEEG of the plurality of brain signals S_{EEG} representing the motor cortical activity of the subject S during said detected muscle activity.

The signals S_{EMG}received are sent to a digitizer block 21 which, by means of a threshold system, processes each acquired electromyographic signal in order to derive a respective digitized signal OOM.

In particular, the digitizer block 21 is configured to derive a respective ON/OFF, muscle activation, binary signal OOM for each signal S_{EMG} corresponding to a respective monitored muscle (channel). The digitizer block 21 is preferably configured to implement a moving-threshold system able to adapt to variations in muscle tone, as will become clearer below.

According to a particularly preferred aspect of the invention, a reference muscle contraction signal MT is generated in response to a contraction of a reference muscle detected by the analysis and processing of one or more of said signals S_{EMG}.

This reference muscle contraction signal MT is in particular generated bilaterally, namely both for a contraction of the reference muscle on the right side and for a contraction of the reference muscle on the left side. As will become clearer in the continuation of the description, each k-th contraction identified by the signal MT is preferably designed to form the elementary timing unit for the analysis and processing steps of the apparatus 20 of the present invention and in particular to act as a trigger for enabling the start of the analysis and processing of the brain signals S_{EEG} by the unit CAU.

An MAP extractor block 22 receives at its input the muscle activation binary signals OOM derived from the signals S_{EMG} and analyses and processes them in order to extract the detected muscle activity pattern MAP.

In the context of the present description, MAP is understood as meaning a muscle activity pattern (profile) representative of the contracted or relaxed physiological condition of the selected muscles monitored by the signals S_{EMG}.

An MAP may in particular be a digital signal or data, in particular a directional data structure such as a vector, extracted from the digitized signals S_{EMG}.

As will become clearer below, the MAP extractor block 22 is preferably configured to extract a muscle activity pattern MAP detected for each (k-th) contraction of a reference muscle, emitting an associated digital flow MAP(k) of consecutive MAPs.

By means of this MAP extraction step, the method allows the analysis of the general muscle state (of all the monitored muscles) over a given time period, in particular during a period spanning the presence of the MT signal indicating a contraction of a reference muscle. The extracted MAP takes into account in fact the contracted state of the selected muscles at the k-th contraction of the reference muscle.

The extracted MAP is sent to an MAP-based scoring block 23 able to generate an indicator MAScore of normality/abnormality for each MAP extracted by the block 22.

The block 23 applies in particular a scoring method (assignment of a score) in order to generate an indicator MAScore(k) which quantifies a degree of similarity between the pattern MAP(k) being analysed and a standard muscle behaviour model, in particular recorded when there is no loss of balance.

For example, the scoring method correlates, in particular compares, the extracted MAP with the standard muscle behaviour model and generates a high MASCore value if the MAP is sufficiently similar to the standard muscle behaviour (normality) or a low MAScore value in the case of an anomalous MAP.

Each MAScore indicator is preferably composed of a scalar value, in particular of between 0 and 1.

Preferred methods for scoring and obtaining a standard muscle behaviour will be illustrated below.

At the output of the MAP-based scoring block there is therefore present a digital flow of indicators MAScore(k) of the normality/abnormality of the MAP which is sent to a threshold decider 24 which outputs a respective flow of binary indicators MA(k) of the normality/abnormality of the detected muscle activity pattern of said muscles of the subject.

This operation enables the digitization of the scalar value of MAScore(k) which is converted into a (boolean) binary indicator MA(k) of the normality/abnormality of the muscle activity pattern (MAP(k) detected at the k-th contraction of a reference muscle. Although not strictly necessary, the use of binary indicators is particularly preferred in order to ensure that the classification step performed by the classifier CL is kept rapid and computationally simple.

According to a preferred embodiment, in the decider 24, the MAScore(k) at the k-th contraction is compared with a statistical threshold associated with the previous history of the muscle indicators MAScore. As a result it is possible to distinguish better between MAscore indicators resulting from ordinary activities, typically tending towards 1, and MAScore indicators associated with losses of balance, typically tending towards zero.

With reference again to FIG. 1, this shows in schematic form a muscle analysis updating unit uMAU which is in particular configured to receive at its input a plurality of previous MAScore(k-x) indicators and calculate an updated threshold for the decider 24 for extraction of the binary indicator MA(k).

Preferably, the updating unit uMAU is also configured to receive at its input a plurality of extracted muscle activity patterns MAP(k) and generate an updated standard behaviour model.

Advantageously, updating of the threshold(s) of the decider 24 may be very rapid and ensures a high degree of reliability of the indicator MA(k) of normality/abnormality of the MAP with respect to the specific activities of ordinary life.

Updating of the standard behaviour model may instead be processed more slowly so as to allow the processing method to be adapted better to the transition from an ordinary activity to another activity which generally occurs in a continuous and not sporadic manner.

Surprisingly the inventors have observed that, even when there is variation in the ordinary activity of the subject S, the MAP-based MAScore indicators associated with a loss of balance are always very low owing to the prolonged and simultaneous contractions of muscles which normally are not contracted together, typical, only of an unstable posture.

The processing method is therefore robust and reliable and the faster synergic updating of the thresholds and slower synergic updating of the standard muscle behaviour model is that which is preferred in order to maximize this robustness and reliability.

With reference still to FIGS. 1 and 3, the detection of a contraction of a reference muscle by the MAU unit generates the reference muscle contraction signal MT which is sent to the cortical analysis unit CAU in order to start the analysis and processing of the plurality of brain signals S_{EEG} received and available on the respective buffers BEEG.

In greater detail, the unit CAU preferably analyses the plurality of signals S_{EEG}representative of the cortical activity of the subject S in a time interval preceding a k-th detected contraction MT of a reference muscle, so as to generate a plurality of cortical response indicators of the subject upon occurrence of said detected muscle contraction.

In an example of embodiment shown in FIG. 3, the cortical analysis unit analyses each of the signals (called also “channels” below) S_{EEG}, so as to quantify a rate of variation in the power of the signals EEG, and extracts in the block 26 a first set of first level cortical response indicators, in particular consisting of a respective cortical response parameter in. Said extraction occurs preferably by means of linear estimation algorithms, in particular least square algorithms. Preferably, each signal S_{EEG} is processed beforehand by means of a sliding-window time-frequency analysis step and/or by means of a band multiplexing step carried out on a plurality of predefined frequency bands of interest.

As will become clearer below, preferably, based on said cortical response parameter In, the CAU processes during a generalization step at least four binary (boolean) indicators of a generalized cortical response, each representative of the normality/abnormality of a generalized cortical response in a respective cortical macro-area. Said cortical macro-areas include, in particular, one or more, preferably all, of the following cortical macro-areas: supplementary motor area, motor area. sensory-motor area and parietal area.

According to a further preferred aspect, the CAU further processes at least one cortical response lateralization indicator which is designed to provide an indication of the involvement of the left and right cortical sides in the cortical activity analysed.

As can be seen from the example schematically shown in FIG. 3, the CAU unit is in this case configured for the generation, upon occurrence of each k-th contraction of a reference muscle, of the cortical response indicators for each channel monitored by the signals S_{EEG} and for each of the plurality of frequency bands of interest, which preferably include one or more, even more preferably all of the following frequency bands: θ (4-7 Hz), α (8-12 Hz), β I, β II, β III (13-15, 16-20, 21-40 Hz).

In the example of FIG. 3, the cortical response indicator \hat{m} is extracted simultaneously for each of the (13) channels and (5) bands of interest, generating (65) respective first-level cortical response indicators in. Moreover, a number (20) of generalized response indicators I_{EG}g equal to the number of cortical macro-areas selected times the number of bands of interest, and a lateralization indicator LF(k) for each band of interest are generated.

With reference still to FIGS. 1 and 3, at each k-th contraction detected, the digital flows of indicators MA(k) and of cortical response indicators are sent to the classifier CL which processes them, correlating them in order to generate a signal Aout indicating an imminent loss of balance, which will be for example a logic signal with a value 1 for each contraction of the reference muscle where an imminent loss of balance was detected, otherwise 0.

Some aspects and preferred embodiments of the processing apparatus and method of the present invention will be described now in greater detail, it being understood that each of the techniques illustrated below may be incorporated in or combined with each embodiment of the invention described here.

It will also be clear to the person skilled in the art that, although described separately, the muscle analysis and cortical analysis steps will take place at least partially in parallel, following activation of the CAU by the signal MT.

Muscle Analysis Unit

Digitizer block S_{EMG}

With reference to FIG. 4, the Muscle Analysis Unit includes a digitization block 21 in which the plurality of signals SEmGX received (10 in the preferred example) are numerically processed (one sample at a time) in order to derive a binary, ON/OFF, muscle activation signal OOMx for each respective monitored muscle. In the following description, x may be replaced by a number which identifies a respective monitored channel/muscle.

In the example shown, when the muscle monitored by the respective signal S_{EMG}X is contracted, the associated signal OOMx=1, otherwise OOMx=0. The procedure is preferably implemented using a moving-threshold approach able to adapt to the changes in muscle tone (for example, owing to fatigue). The step of digitization of the ON/OFF muscle model (FIG. 3) generates 10 parallel signals OOM1, OOM2, . . . , OOM10, which are sent to the block 22 for extraction of the Muscle Activity Pattern MAP.

Two signals of the ten digitized signals SEMGx are selected as reference muscle contraction signals MT for enabling the cortical unit, acting as a trigger (in the example shown in FIG. 4, OOM2=MT). These signals MT are derived from the same muscle, bilaterally.

It is particularly preferred to select a muscle which uniquely identifies a specific phase of the walking action, allowing the exclusion, from the analysis, of the cortical activity which is not strictly related to the specific movement, thus ensuring protection from false alarms in the cortical analysis unit.

A preferred muscle is the Lateral Gastrocnemius, whose sensors for acquisition of EMG signals are indicated by R_LG and L_LG in FIG. 2. A contraction of the Gastrocnemius identifies, in fact, uniquely the double support phase and is therefore particularly recommended for use in a method according to the invention as a muscle contraction reference signal MT.

MAP Extractor Block

As shown in FIG. 4, in the Muscle Analysis Unit MAU, the 10 parallel binary signals OOM1, MT, OOM3 are sent to the MAP extractor block 22 which is configured to analyse the general muscle state (of all the muscles analysed) within a specific time interval spanning the occurrence of each reference contraction signal MT.

The block 22 emits a muscle activity pattern in the form of a vector MAP(k) comprising a corresponding number of elements (10 in the example). Each element corresponds to an evaluated muscle and assumes the value 1 if the muscle is active for more than half of a predefined observation period, otherwise it is equal to 0 (time predominance rule).

MAP-Based Scoring Block

As described above, the MAP-based scoring block 23 is designed to receive at its input the digital flow of extracted patterns MAP(k) and to process a respective indicator MAScore(k) quantifying the normality/abnormality of each MAP(k).

As shown in FIG. 4, the vector MAP(k) is in particular correlated with a standard muscle behaviour SMB model in order to calculate a respective MAP-based normality/abnormality score MAScore(k).

Standard Muscle Behaviour Model

According to a preferred embodiment, the standard muscle behaviour model SMB comprises a set of weights associated with the contraction of each muscle monitored during a walking phase which is undisturbed and/or without loss of balance, in particular monitored during a plurality of ordinary activities which are undisturbed and/or without loss of balance.

In particular, during an initial calibration phase, the MAPs resulting from data acquired in absence of a loss of balance/during undisturbed walking may be collected and statistically analysed in order to extract weights associated with the contraction of each monitored muscle. This set of weights form the SMB model.

The SMB model may be preferably in the form of a directional data structure, in particular one or more weight vectors.

In a preferred embodiment, the SMB model includes two weight vectors, one derived from the movements of the right leg and one derived from the movements of the left leg, so that problems of asymmetry may be advantageously avoided, improving the reliability of the method.

In this way it is possible to extract the most probable muscle model and, consequently, a score assignment method which provides a high score if the MAP(k) to be classified is similar to the standard behaviour model or a low score if there are anomalies such as a loss of balance.

With reference still to FIG. 4, the binary vector MAP(k) extracted at the k-th contraction is received by the MAP-based scoring block 23 which processes it according to the statistical weight SMB vectors, assigning a score MAScore(k) which tends towards 1 if the MAP(k) to be classified is similar to the standard muscle, otherwise it tends towards 0.

In particular, in order to determine the MAScore, the block 23 relates, element by element, the MAP(k) vector to the weight vector correlated with it. For example, the MAP(k) extracted at a k-th contraction derived from the right gastrocnemius is correlated and processed depending on the weight vector associated with the standard muscle behaviour of the right leg.

Particularly preferred examples of a method for calculating an MAP-based score MAScore are described below.

Preferred Example of an MAP(k)-Based Score

Let us assume by way of example an MAP(k), namely the MAP vector extracted at the k-th reference muscle contraction comprising 10 elements (∈ [symbol] ^{10,1}), where there are 10 muscles monitored by the respective signals S_{EMG}. FIG. 5 shows in schematic form an MAP-based scoring block which shows some steps of the method for calculating an MAScore(k).

With reference to FIG. 5 a MAP[1 . . . 10] at the k-th contraction has for example the following composition:

Muscle 1 contracted→MAP(1)=1

Muscle 2 contracted→MAP(2)=1

Muscle 3 relaxed→MAP(3)=0

Muscle 9 contracted→MAP(9)=1

Muscle 10 contracted→MAP(10)=0

The MAP(k) is correlated with the standard muscle behaviour (weight vector) and with its complementary (complementary weight vector according to the statistical definition). E.g., if the weight vector (1)=0.8, Negated Weight Vector (1)=1-0.8 =0.2.

In greater detail:

23a: the MAP(k) is multiplied one element at a time for the Weight Vector and

23b: all the elements of the resultant vector (∈ [symbol] ^{10,1}) are added together, generating a first scalar 23c;

23d: the negated MAP(k) is simultaneously multiplied, one element at a time, for the Negated Weight Vector and

23e: all the elements of the resultant vector (∈ [symbol] ^{10,1}) are added together, generating a second scalar 23f.

The two scalars are then added together 23g and are normalized 23h at the maximum possible score.

The normalization transforms the vectors involved into a single scalar.

According to a further preferred embodiment, the following are schematically defined:
MAP(i): i-th element of the MAP considered
M̅A̅P̅(̅i̅)̅: i-th element of the MAP negated.
F₁: weight vector
F₀: complemented weight vector (1-F₁)

The score MAScore may be defined as follows:

$$MAScore = \frac{{\sum }_{i = 1}^{10} MAP ( i ) * {F}_{1} ( i ) + {\sum }_{i = 1}^{10} \overline{MAP ( l )} * {F}_{0} ( i )}{{\sum }_{i = 1}^{10} max ( [ {F}_{1} ( i ) , {F}_{0} ( i ) ] )}$$

where max ([F₁(i), F₀(i)]) is the greatest value of the weight F₁(i) and F₀(i). For example, if F₁(i)>F₀(i) for the i-th step the value of F₁(i) will be chosen. The MAP-based score calculation methods which envisage the duplication of the MAP (MAP negated or not) during the correlation with the standard behaviour model are particularly preferred because they allow the statistical weights of both logic states (i.e., both “1” and “0”) to be considered. Otherwise, the informative contribution of the “0” states would be lost, owing to the multiplication of the weights in F0 by 0.

MA(k) Binary Indicator Generator Decider

As described above, the MASCore(k) relating to a k-th contraction of a reference muscle is preferably compared with a threshold in a decider 24 in order to generate a binary indicator MA(k) of normality/abnormality of the associated extracted MAP(k). The threshold is in particular able to distinguish between MAScores resulting from undisturbed contractions and MAScores typical of postural instability situations, so that the decider 24 generates for example an MA(k)=1 in the presence of an anomaly in the muscle activity at the k-th contraction of the MT.

For this purpose, according to a preferred embodiment, the method envisages updating the decision threshold for generation of the indicator MA(k) by means of the updating unit uMAU, which may be configured to consider the MAScores resulting from an observation window prior to the k-th contraction being analysed, these being for example inserted in a prior score vector.

The unit uMAU derives statistically the updated threshold from the prior score vector, for example as the 5th percentile of the data contained in the vector. The updated threshold is then set in the decider 24 and the MAScore is then compared with this updated threshold.

Cortical Analysis Unit

With reference to FIGS. 3 and 4, once enabled by means of the trigger signal MT indicating a contraction of a right or left side reference muscle, the cortical unit CAU extracts, from the 13 circular buffers BEEG, respective data blocks S_{EEG}′ included in a time window preceding the occurence of the respective muscle contraction reference signal MT which enabled the cortical analysis. A preferred range of the time window for extraction of the data blocks S_{EEG}′ is between 700 ms and 900 ms, in particular 800 ms (corresponding to 400 samples with a previously described acquisition profile), preceding the reference contraction MT of the reference muscle.

The choice of this preferred window for the data blocks S_{EEG}′ ensures advantageously the presence of information on the reactive cortical response also in bands which react very rapidly to a disturbance in the equilibrium, such as in particular the band 0 (peak estimated at ˜185 ms from the start of the disturbance), avoiding at the same time the introduction of undesirable computational delays.

This preferred time window is particularly suitable for use with a reference contraction signal resulting from a lateral gastrocnemius, the contraction of which on the loss-of-balance leg side occurs statistically at ˜325 ms from the loss of balance. It will be clear to the person skilled in the art that a different time window may be possible depending on the reference muscle chosen and/or the EEG bands monitored.

This subset S_{EEG}′ of EEG data may be subjected preliminarily to on-line artefact rejection processing. In this connection, this stage may be for example entrusted to the Riemaniann algorithm “Artifacts Subspace Reconstruction (rASR)” described in Blum S, Jacobsen NSJ, Bleichner MG and Debener S (2019) “A Riemannian Modification of Artifact Subspace Reconstruction for EEG Artifact Handling.” (Front. Hum. Neurosci. 13:141. doi: 10.3389/fnhum .2019.00141).

The cortical unit CAU analyses the brain data blocks S_{EEG}′ without artefacts, quantifying a rate of variation in the power of the signals S_{EEG}.

In particular, measurements of the power spectral density (PSD) are performed by means of a time-frequency analysis block 26a based on a sliding window Fast Fourier Transform (FFT).

A band-multiplexing section 26b is placed downstream of the time-frequency analysis block 26a and is designed to extract a spectral contribution in one or more bands of interest, in particular in the following five preferred bands of interest involved in the reactive cortical response in losses of balance: θ(4-7 Hz), α (8-12 Hz), β I, β II e β III (13-15, 15-20, 21-40 Hz).

In particular, the Band Multiplexing section 26b adds together all the spectral contributions from the FFT block 26a which fall within a specific band of interest, in order to define the total spectral contribution thereof.

The data Y from the band multiplexing block 26b is sent to a linear estimation block, in particular based on the least squares, which extracts the cortical response parameter for a plurality of EEG channels (13) in the specific bands of interest (5).

In greater detail, in the example shown, the subset S_{EEG}′ of EEG data is subdivided, 26a, into a plurality of superimposed windows in predefined number and length, L_{win} (for example 20 windows of L_{win}=200 samples superimposed at a 10 sample interval). These windows are configured to cover the entire subset S_{EEG}′ of data (e.g. 400 samples=800 ms). Considering a single EEG window, the block 26a performs an FFT, in the example of FIG. 4 with a spectral resolution of 2.5 Hz (considering fs_{EEG}=500 samples/s and L_{win}=200 samples).

For each window analysed the band multiplexing section 26b extracts a matrix S_{Bol} ∈ R^{nch, nBol}, with n_{ch}=13 number of EEG channels monitored and nBol=5 number of bands involved in the band multiplexing.

The calculation of the S_{Bol} is then extended to the 20 windows analysed, generating a three-dimensional matrix: Y∈ R^{nch,nBol,nW} with nW=20 windows analysed.

The data Y from the band multiplexing section 26b and relating to the 20 windows analysed are finally sent to the linear estimation unit 26c which extracts linear models of these progressions by means of least squares fitting. In particular, this cortical response estimate may be based on the easy approximation which the brain response, described by the 20 points on the i-th channel and j-ith band of interest, may assimilate with a linear model of the type x(t)=m·t+q affected, however, by measurement error. In this context, the model of the extracted parameters (\hat{p} in FIG. 4) contains the intercepted estimate of the linear model \hat{q}=\hat{p}Ii_|_{Bol}[1], as well as the gradient of the straight line resulting from the estimated approximation of the narrow line m\hat{ }=\hat{m}=\hat{p}(i)|_{Bol}[2,]. The linear models obtained (OLS Estimate of the PSD—FIG. 4) allows approximation of the cortical response parameter as the gradient, in, of the model for the channel and the band of interest. This estimate based on the OLS is at the same time applied to the 13 channels and to the 5 bands of interest, generating 65 values of \hat{m}.

In the same formula, shown in FIG. 4, A is the matrix of the base functions containing a column of 1 and a time vector column (t=20: 800 ms, interval 20 ms)

Generalization

With reference to FIGS. 3 and 6, the generated cortical response indicators in are processed by means of a so-called generalization procedure 27a.

Said generalization, in accordance with that shown in FIG. 6, preferably comprises averaging a plurality of said cortical response indicators ^{\hat{m}ch}1, band, . . . ,\hat{m}ch3,band extracted by the analysis of the individual channels S_{EEG}, so as to obtain a smaller number (20 in the example) of generalized cortical response indicators, each of which takes into account predefined monitored EEG channels belonging to a respective broad cortical area. These broad cortical areas, also known as functional macro-areas, preferably comprise: supplementary motor area (SMA), motor area (M1), sensory-motor area (S1) and parietal area (PPC).

In particular, in the preferred configuration for acquisition of signals S_{EMG}described with reference to FIGS. 1-2, the channels belonging to the said four functional macro-areas are:
Supplementary motor area (SMA); F3, Fz, F4;
Motor area (M1): C3, Cz, C4;
Sensory-motor area (S1); Cp5, Cp1, Cp2, Cp6;
Parietal area (PPC): P3, Pz, P4.

It is clear that this is only a preferred example of generalization and that further channels or a subset of channels selected here may be used for each macro-area. It is also possible to select only one, two or more functional macro-areas from among those proposed or also different macro-areas, depending on the degree of robustness, reliability and response speed which the designer requires for the processing method and apparatus.

The generalization step provides indicators for qualitative control of the general cortical involvement of the subject and allows a reduction of the data to be analysed for classification (in the example 65 values of \hat{m} obtained from 13 channels and 5 bands of interest are reduced to 20 indicators, each of which refers to a respective average parameter \hat{m} of a respective macro-area and band of interest. Each element forming the vector may be identified with the notation \hat{m}_{macro-area,band} (e.g. \hat{m}_{SMA,α} identifies the generalized cortical response m at the supplementary motor macro-area, acquired and evaluated in the band of interest α).

As shown in FIGS. 3 and 6, a particularly preferred implementation involves the conversion 27b of each generated generalized indicator \hat{m}_{macro-area,band} into a binary indicator b\hat{m}_{macro-area,band} of generalized cortical response, by means of a threshold system. The set I_{EG}g of these 20 binary generalization indicators is sent to the classifier CL (FIGS. 3, 4 and 8).

Although not strictly necessary, the use of binary indicators is particularly preferred in order to ensure that the classification step performed by the classifier CL is kept rapid and computationally simple.

Advantageously, the threshold system may be of the moving threshold type, able to adapt the threshold to variations of the cortical response parameters over time.

In the exemplary diagrams shown, this is schematically represented by an updating unit uCAU for the cortical analysis, designed to receive at its input a plurality of previous cortical response parameters and process them statistically in order to provide the CAU unit with updated thresholds.

For example, for each cortical response indicator, the last N generalized cortical response parameters N generated are sent to the unit uCAU and a respective dedicated vector is constructed, said vector being statistically analysed in order to determine respective thresholds of the system 27b; these thresholds are for example determined as the 95th percentile of the vector analysed. Preferably, the thresholds for the binary indicators of the generalized cortical response are updated at each k-th contraction of the reference muscle.

Lateralization

With reference to FIGS. 3 and 7, the generated cortical response indicators \hat{m} are furthermore processed by means of a so-called lateralization procedure 28.

During the lateralization step, in accordance with what shown in FIG. 3, a selection of said cortical response indicators, derived from the channels lateral with respect to the middle cortical line, is preferably processed, in particular averaged, so as to derive two overall lateralized cortical response parameters RS(k), LS(k), i.e. a right side parameter and left side parameter.

As shown, preferably, the overall lateralized cortical response parameters RS(k), LS(k) are further processed jointly in order to obtain a respective binary lateralization indicator LF(k).

In a similar manner to that envisaged for the other indicators of the present invention, the lateralization binary indicator LF(k) is preferably calculated for each k-th contraction of the reference muscle and for each band of interest of the cortical analysis.

In the preferred example shown, it is envisaged that in stage 28a for obtaining the binary lateralization indicator the parameters RS(k), LS9k) are preferably correlated. The result of the relation in terms of absolute value is then compared with a quantity 1_{+ε}, where ε is an empirically derivable tolerance. This operation generates a binary lateralization indicator LF(k) band for each band of interest.

The lateralization step advantageously allows an evaluation, during classification, of the incidence of the increase on a specific (left or right) side, analysing the relation between two specific macro-areas: the left side containing {F, C, P}3 and the mean {Cp1, Cp5} and the right side which involves {F, C, P}4 and the mean {Cp2, Cp6}.

In fact, during a reactive response, the cortical involvement is normally widespread. During the undisturbed phases, instead, the cortical response tends to be more lateralized. For example, during walking there will be a greater increase in the cortical activity in the ipsilateral motor area with double supporting of the foot (namely upon contraction of the reference muscle identified by the signal MT).

Classifier

With reference to FIG. 8, a preferred embodiment of a classifier CL for use in a method according to the present invention is now described.

As shown in FIG. 8, at each k-th contraction, the boolean indicators derived from the cortical response generalization section, the boolean indicators derived from the cortical response lateralization section and the binary indicator MA(k) derived from the unit MAU are sent to the classifier CL.

The classifier CL is preferably a logic classifier, in particular in the form of a logic network with at least three levels, for example composed of a family of comparators.

Said at least three levels are preferably configured to verify the following conditions: (i) anomalies in the generalized cortical response (presence of an increase in the cortical response in several macro-areas), (ii) presence of anomalous non-lateralized cortical responses (anomaly not concentrated on one side) and (ii) simultaneous presence of the binary indicator MA(k) indicating anomalies of the MAP.

In said specific condition, the classifier generates a signal Aout indicating an imminent loss of balance (e.g. which places the signal Aout in the logic state indicating detection and imminence of a loss of balance).

With reference still to FIG. 8, the first level considers the binary alerts from the 4 cortical macro-areas (e.g. b\hat{m}_{SMA,α}(i)(k)), in all the bands of interest). The classifier CL checks in a first classifier stage CL1a, CL1b the presence of a widespread increase in the power of the brain signal. In particular, if for each band assessed more than (>) a predefined number, in particular more than 2 cortical macro-areas, are affected by the increase in power, indicated by the respective indicator, the classifier CL sets by means of an adder-comparator node CL1a a generalization flag (GF_{α}(k)) to 1; otherwise it sets it to 0.

The classifier CL therefore analyses the generalization flags (GF) in all the bands of interest, by means of a respective adder-comparator node CL1b configured so that, if more than (>) a predefined number, in particular more than 2 bands of interest, are involved in the increase in cortical activity, an output flag F1(k) of the 1st classification level CL1a,b is set to 1.

The 2nd level CL2 incorporated by the classifier CL analyses a ratio between the left cortical side and the right cortical side (x/y).

As shown in FIG. 8, the classifier CL may receive at its input directly the right side lateralization indicators LS(k) and left side lateralization indicators LS(k), but it is clear to the person skilled in the art that these indicators may be converted into a binary indicator LK(k) already in the brain analysis unit CAU, as described above with reference to FIGS. 3,7.

If the ratio is greater than 1+ε or less than 1−ε, where ε is the specific tolerance which may be empirically derived (−), a lateral increase in recorded.

The second classifier level CL2 of the classifier CL generates in particular a binary flag LF_{α}(k), which is set to 1 if a lateralized brain activity is recognized. In the example, an adder-comparator node CL2 receives at its input all the binary lateralization indicators LF(k) and, if less than (<) a predefined number, in particular less than 2 LF(k), are set to 1, it sets a second level flag F2(k) to 1; otherwise, it sets it to 0.

If both the 1st and 2nd level flags F1, F2 are ON(1), this means that the system has detected a widespread non-lateralized increase of the cortical activity. The flags F1(k) and F2(k) are sent to a third classifier stage CL3, for example formed by an AND logic gate which also receives at its input the indicator MA(k) of normality/abnormality of the MAP at the k-th reference muscle contraction.

As may be deduced, the third classifier stage CL3 is configured so that, if an anomalous muscle behaviour together with a widespread and non-lateralized cortical behaviour are detected, the k-th contraction of the reference muscle MT is classified as resulting from a potential loss of balance and the respective signal Aout(k) indicating an imminent loss of balance is set to 1.

As previously described, the classification output Aout of this logic network may be used to enable a fall prevention strategy (e.g. through wearable robotics and exoskeletons).

It is therefore clear how a processing method and apparatus according to the invention allow the accurate detection and signalling of an imminent involuntary loss of balance and thus a likely imminent fall through the synchronized analysis and processing of electrophysiological signals.

The detection and signalling may advantageously be performed substantially in real time.

The method and the apparatus allow in particular the processing of information derived from the electromyographic (EMG) profiles of the subject, in order to analyse the muscular activity performed and, at the same time, analyse the brain signals, acquired by means of electroencephalography (EEG) and preceding the motor activity, thus allowing the analysis of the cortical involvement of the subject during a reactive response or during normal motor planning (e.g. undisturbed walking, orthostatic position-chair transition, bends, overcoming an obstacle, etc.).

Further advantageous aspects of the invention result from the fact that:
The processing method is computationally low-intensive, capable of analysing in the time and frequency domain the reactive cortical dynamics (at the scalp level) involved in postural adjustment processes, when the (static or dynamic) equilibrium is unexpectedly disturbed by unexpected events of varying nature.
The method can implement a multiple control system which verifies the simultaneous presence of “non-standard” neuro-muscular dynamics so as to greatly reduce false alarms by guaranteeing a high specificity and robustness in respect of ordinary life activities.
The algorithm advantageously takes into account physiological considerations in both the neural and the muscular sphere, obtaining complete control of a variety of fall typologies of an involuntary nature, allowing, among other things, the replacement of Motion Capture Systems for the recognition of induced slipping falls.
The robustness of the processing method is preferably ensured by a continuous automatic recalibration procedure during use, capable of adapting to the circadian rhythm of the user subject. In particular, one or more thresholds, and/or a standard muscle behaviour model, and/or a MAP-based scoring method may preferably be updated by means of the method and apparatus according to the invention.
The method and apparatus allow high accuracy values (>95%) to be achieved, while keeping detection times within the limit imposed for implementing compensatory action. For example, with a classifier using binary/boolean indicators, it is easy to generate a signal indicating imminent loss of balance within 550 ms from the start of the fall event. In particular, experimental tests have shown that the method of the invention using a three-level logical classifier as described with reference to FIG. 8 is able to provide a signal indicating an imminent loss of balance in about 370 ms.
The fall prevention system of the invention may be advantageously fully wearable, in particular owing to an acquisition unit with wireless transmission means and/or the fact that the processing method can be easily implemented and integrated in any programmable electronic device (e.g. microcontrollers, FPGAs, or even smartphones).

The apparatus according to the invention is in fact easy and inexpensive to implement and produce by means of simple configuration and programming steps within the competence of the average person skilled in the art.

Detailed Example of Processing Method

For the sake of completeness of the description, below a detailed non-limiting example of a complete processing cycle performed by a preferred embodiment of the fall prevention detection system which includes an apparatus according to the present invention is provided.

First use (k=1);

1) Provision of the acquisition unit 10 with EEG sensors 12 and EMG sensors 11 arranged and configured according to the preferred embodiments described with reference to FIGS. 1 and 2; and

2) Activation of the processing unit 20 which receives and stores in the circular registers BEEG,BEEG the data sent wirelessly from the acquisition unit 10;

3) During the double support of the first step taken by the subject S being monitored, the gastrocnemius (reference muscle) contracts, generating the first reference contraction (k=1);

4) The muscle analysis unit MAU digitizes the received signals S_{EMG} (block 21) which are forwarded to the pattern extraction block 22 MAP(k=1) and at the same time derives from the digitized reference muscle signal MT the contraction line k=1 of the reference muscle which is sent as a trigger signal MT to the cortical analysis unit CAU.

5) The operations of the muscle analysis unit and the cortical analysis unit shown below will be performed in parallel. For greater clarity the operations derived from the same unit will be clarified en bloc.

6) Muscle analysis unit MAU: the MAP(k=1) is extracted from the block 22 and saved in a dedicated memory location for future processing.

7) Cortical analysis unit CAU: activated by the leading edge of the reference muscle signal MT, the content of the circular register BEEG containing the brain signal S_{EEG} prior to the contraction k=1 detected is extracted. Considering this subset of data the cortical analysis unit CAU extracts 65 cortical response parameters \hat{m} (13 EEG channels*5 bands) for the first contraction k=1.

8) Cortical analysis unit CAU: the 65 cortical response parameters \hat{m} are sent to a generalization routine 27a (FIG. 6). This step generates 20 generalized scalar cortical response parameters (4 macro-areas*5 bands)

9) Cortical analysis unit CAU: 20 indicators relating to the generalization step are saved in a dedicated memory location for future processing.

Calibration Step (First Use): k=EoC−EoC: Calibration End:

10) The method steps indicated in points 2-9 are cyclically repeated EoC times, where EoC is an empirically derivable number of contractions necessary for initial calibration.

11) After collecting the parameters according to points 6 and 9, starting from the end of contraction k=EoC, the processing unit 20 starts a step for extraction of the statistical thresholds in order to determine the binary indicators of MAP normality/abnormality (preferably performed by the unit uMAU) and of cortical response (preferably performed by the unit uCAU) to be sent to the classifier.

Statistical Threshold Extraction

12) Muscle analysis unit MAU: the MAP(k=1, . . . , EoC) are statistically analysed in order to determine the weights which form the standard muscle behaviour model. The weights are based on the statistical contraction (activation) occurrence for each of the muscles monitored upon contraction of the reference muscle.

For example if the first element of the MAP patterns was twice in a high logic state (contracted muscle) on EoC=10 MAPs analysed, the relative weight will be 0.2 (20%). In the same way, if a muscle is instead normally contracted its weight will tend towards 1.

13) Muscle analysis unit: the last Nobs (where Nobs<EoC) of the MAP patterns collected (MAP(k=EoC-Nobs . . . EoC)) are considered. In accordance with that shown in FIG. 5, each of the MAPs is individually multiplied, one element at a time, for the statistical weights according to the preceding point and the result is standardized to the maximum score which can be obtained.

The set of these analysed score Nobs (score vector) is in turn statistically analysed in order to determine the first threshold of the system (it is updated again at each k-th contraction for k>EoC). As already mentioned, this threshold is, for example, determined as the 5th percentile of the score vector.

14) Cortical analysis unit CAU: the last Nobs 20 generalized cortical response parameters according to point 9 are considered. For each of these parameters a dedicated vector is constructed and statistically analysed in order to determine the first thresholds of the system (they will be updated again at each k-th contraction for k>RoC). These thresholds are determined, for example, as the 95th percentile of the vector analysed.

15) The overall procedure generates 26 initial thresholds, to be applied from the contraction k=EoC+1: 25 thresholds dedicated for the cortical analysis unit, 1 threshold dedicated for the muscular analysis unit.

Generic Use (k>EoC)-k-th Contraction:

16) During the double support of the first step taken by the subject S being monitored, the gastrocnemius (selected reference muscle) contracts, generating the k-th contraction.

17) The muscle analysis unit MAU digitizes the S_{EMG} monitored and forwards them (OOM) to the block 23 for extraction of the pattern MAP(k). At the same time the unit MAU derives the contraction line of the reference muscle, generating the signal MT which is sent as a trigger for starting the cortical unit.

18) The operations of the muscle analysis unit and the cortical analysis unit shown below will be performed in parallel. For greater clarity the operations derived from the same unit will be clarified en bloc.

19) Muscle analysis unit MAU: the MAP(k) is multiplied by the weight vector using the methods indicated in point 13. The resultant score of the standardization process (MAScore(k) is compared with the threshold for the k-1 th contraction. If the MASscore(k) is less than this threshold a binary indicator of an anomaly of the muscle pattern MA(k)=1 is generated, otherwise MA(k)=0 indicating a situation of normality consistent with the standard muscle behaviour.

20) Cortical analysis unit CAU: activated by the leading edge of the reference muscle contraction signal MT, the content of the circular register BEEG containing the brain signal to the contraction is extracted. Considering this data subset (S_{EEG}′), the processing unit extracts 65 cortical response parameters (13 channels EEG*5 bands).

21) Cortical analysis unit CAU: the 65 cortical response parameters are sent to a routine which implements the generalization step (FIG. 6) and a lateralization step (FIG. 7). These procedures generate respectively, 20 generalized cortical response parameters (4 macro-areas*5 bands) and 5 lateral cortical response parameters.

22) Cortical analysis unit CAU: The 20 generalized cortical response parameters, according to the preceding point, are compared with the respective thresholds calculated at the k-1 contraction. If these parameters are greater than this threshold a boolean anomaly parameter for the generalized cortical response in the specific macro-area and in the specific band of interest is generated.

23) Cortical analysis unit CAU: the 5 lateralized cortical response parameters obtained from the comparison between the right-side cortical response value and left-side cortical response value are compared with a quantity 1 +ε, where ε is the empirically derivable tolerance, in order to obtain a respective binary lateralization indicator LF_{band}(k) (FIG. 8).

24) Logic classifier: in accordance with that described with reference to FIG. 8, the classifier CL receives at its input the following parameters: 20 boolean parameters derived from the cortical response generalization section (I_{EG}g), 5 boolean parameters derived from the cortical response lateralization section (LFⱼ(k) in FIG. 4 and 1 binary indicator MA(k) of the normality/abnormality of the MAP(k). The classifier is formed by a 3-level logic network composed of a family of comparators.

25) Logic classifier CL: The first of the three levels checks for the presence of an anomalous increase in the response in several macro-areas and in several frequency bands. In each single band, if at least an anomalous increase of the cortical response parameter is registered in 3 macro-areas, the dedicated flag GF_{band}(k) is set to 1. If this increase extends to at least 3 bands of interest the first cortical alert flag F1(k) is set to 1 (FIG. 8.).

26) Logic classifier CL: The second of the three levels analyses the presence of anomalies in the lateralized cortical response. If less than two bands are affected by the lateralization (LF_{band}(k)) (lateralization absent) the second cortical alert flag F2(k) is set to 1 (FIG. 8).

27) Logic classifier CL: The third logic level analyses the simultaneous presence of all the alert flags: MA(k), F1(k) and F2(k). If they are all set to a value indicating an anomaly (“1” in the example), namely, if the following situation occurs: (i) anomalies in the generalized cortical response (present in several macro-areas), (ii) presence of non-lateralized anomalous cortical responses (the anomaly must not be concentrated on one side) and (iii) non-standard muscle behaviour (MA(k)=1, the classifier generates or sets the signal Aout indicating an imminent loss of balance to a corresponding logic state (“1”).

28) Any signal Aout indicating imminent loss of balance is preferably sent to the unit for implementing a corrective or preventive action, consisting for example of a wearable robotic system.

29) The mechatronic implementing system performs, if necessary, corrective action aimed at preventing falling of the subject, in response to said signal indicating an imminent of loss of balance.

Although described in connection with a number of embodiments and a number of preferred examples of implementation of the invention, it is understood that the scope of protection of the present patent is determined solely by the claims below.

1. A method of processing physiological signals (S_{EMG}; S_{EEG}) acquired from a subject (S), for detecting an imminent loss of balance of the subject and generating a signal (Aout) indicating the imminent loss of balance, comprising the steps of:
reception of a plurality of electromyographic signals (S_{EMG}) representative of a detected muscle activity of a plurality of selected muscles of the subject;
reception of a plurality of brain signals (S_{EEG}), acquired by means of electroencephalogram and representative of a cortical activity of the subject during said muscle activity;
analysis and processing of said plurality of electromyographic signals (S_{EMG}) in order to extract at least one (MAP(k)) muscle activity pattern, MAP, for the detected muscle activity, and generate at least one indicator (MAScore(k); MA(k)) of normality/abnormality of the detected muscle activity pattern;
analysis and processing of said plurality of brain signals (S_{EEG}) in order to generate a plurality of cortical response indicators (I_{EG}g; LF(k)) for the cortical response of the subject upon occurrence of said detected muscle activity;
classification, wherein at least one indicator (MA(k)) of MAP normality/abnormality and one or more of said cortical response indicators are correlated to generate a signal (Aout) indicating an imminent loss of balance;
wherein the cortical response indicators for the cortical response of the subject used in the classification step include at least one indicator of normality/abnormality of the cortical response generalized over one or more cortical macro-areas of the subject upon occurrence of said muscular activity, and an indicator of lateralization of the cortical response, which indicates a normality/abnormality of the involvement of the left and right cortical sides in the cortical response;
and wherein, in the classification step, a signal (Aout) indicating an imminent loss of balance is generated if at least one anomaly in a generalized cortical response over one or more cortical macro-areas, a presence of a non-lateralized anomalous cortical response and a simultaneous abnormality of the muscle activity pattern are detected.

2. The method according to claim 1, wherein each electromyographic signal received is digitized by means of a threshold system in order to obtain a corresponding binary signal (OOMx; MT) of muscle activation for a respective selected muscle, wherein preferably the threshold system (21) is a moving threshold system configured to adapt to changes in muscle tone.

3. The method according to claim 1, wherein the plurality of electromyographic signals (S_{EMG}) includes signals representative of a muscle activity detected, bilaterally, from one or more, preferably all, of the following muscles of the subject: Anterior Tibial (AT), Lateral Gastrocnemius (LG), Vastus Medialis (VM), Rectus Femoris (RF) and Biceps Femoris (BF).

4. The method according to claim 1, wherein the MAP pattern is extracted taking into account the contraction state of the selected muscles upon contraction of a reference muscle.

5. The method according to claim 1, comprising correlating, in particular comparing, an extracted muscle activity pattern MAP with a standard muscle behaviour model, to generate an indicator of MAP normality/abnormality.

6. The method according to claim 5, comprising quantifying with a scoring method a degree of similarity between the detected muscle activity pattern (MAP(k)) and the standard muscle behaviour model in order to obtain a score (MAScore) of normality/abnormality of the detected muscle activity pattern, wherein the score (MAScore) is preferably a scalar value.

7. The method according to claim 1, wherein, for the classification step, at least one binary indicator (MA(k)) of MAP normality/abnormality is generated, wherein the binary indicator (MA(k)) of normality/abnormality of the detected muscle activity pattern is preferably obtained from the score (MAScore) which quantifies a similarity between the detected muscle activity pattern (MAP(k)) and the standard muscle behaviour model, in particular by comparison with a statistical threshold, the threshold being preferably linked to the previous history of the scores (MAScore) of normality/abnormality of the muscle activity pattern.

8. The method according to claim 1, wherein the standard muscle behaviour model is generated from a plurality of MAP muscle activity patterns obtained from signals acquired in absence of a loss of balance, which MAPs are preferably collected and analysed statistically in order to extract a set of weights related to the occurrence of contraction of each selected muscle.

9. The method according to claim 1, wherein the standard behaviour model (SBM) is updated periodically based on a plurality of previously extracted muscle activity patterns.

10. The method according to claim 1, wherein at least two indicators of normality/abnormality of the subject's generalized cortical response to said muscular activity, preferably at least three or four generalized cortical response indicators, each representative of the normality/abnormality of a generalized cortical response over a respective cortical macro-area, are used in the classification step.

11. The method according to claim 10, wherein said cortical macro-areas include one or more, preferably all, of the following cortical macro-areas: supplementary motor area, motor area, sensory-motor area and parietal area.

12. The method according to claim 1, wherein the brain signals (S_{EEG}) include a plurality of signals each obtained from a channel for monitoring the motor area, supplementary motor area and/or sensory-motor area, preferably from at least thirteen channels, in particular two or more and preferably all of the following channels: F3, Fz, F4, C3, Cz, C4, Cp5, Cp1 Cp2, Cp6, P3, Pz and P4.

13. The method according to claim 1, wherein each brain signal (S_{EEG}) received is preliminarily processed by means of a time-frequency analysis with sliding windows and/or by means of band multiplexing in a plurality of predefined frequency bands of interest, wherein the bands of interest include one or more, preferably all, of the following frequency bands: θ (4-7 Hz), α (8-12 Hz), β I (13-15 Hz), βII (16-20 Hz), and β III (21-40 Hz).

14. The method according to claim 1, wherein a first level cortical response indicator (\hat{m}) is extracted for each channel monitored by the brain signals (S_{EEG}) and preferably for each frequency band of interest, wherein extraction is performed preferably by means of a linear estimation algorithm, in particular least squares algorithm.

15. The method according to claim 14, wherein a lateralization indicator is generated from said extracted first level cortical response indicators (\hat{m}), wherein in particular two overall cortical response parameters, of the right and left side respectively, are derived from the first level cortical response indicators respectively extracted from channels on the right side and left side with respect to the median cortical line, and wherein the lateralization indicator is preferably generated based on the value of a ratio between said right side and left side overall cortical response parameters.

16. The method according to claim 1, wherein the one or more generalized cortical response indicators and/or the at least one cortical response lateralization indicator used in the classification step are binary indicators and/or are generated for each band of a plurality of frequency bands of interest.

17. The method according to claim 1, wherein the classification step is carried out by a logical classifier with at least three levels, wherein a signal indicating an imminent loss of balance is generated if a first level (CL1) detects a presence of anomalies in a generalized cortical response in one or more macro-areas, a second level (CL2) detects a presence of one or more abnormal non-lateralized cortical responses and a third level detects a simultaneous abnormality of the muscle activation pattern.

18. The method according to claim 1, wherein the cortical response indicators, the at least one indicator of MAP normality/abnormality and/or said signal (Aout) indicating an imminent loss of balance are generated for each contraction of a reference muscle detected by the analysis and processing of one or more of said electromyographic signals (S_{EMG})

19. The method according to claim 1, comprising detecting, by means of analysis and processing of one or more of said electromyographic signals (S_{EMG}), one or more contractions of a reference muscle among the selected muscles, and defining a reference muscle contraction signal (MT) such that each k-th contraction detected identifies an elementary timing unit for the analysis and processing of electromyographic signals (S_{EMG}) and brain signals (S_{EEG}) and/or for said classification; wherein preferably the reference muscle contraction signal is generated bilaterally for both a right side reference muscle contraction and a left side reference muscle contraction and/or the reference muscle is the lateral gastrocnemius.

20. The method according to claim 19, wherein the analysis and processing of the plurality of brain signals (S_{EEG}) is initiated by the reference muscle signal (MT) generated in response to a contraction of the reference muscle detected by the analysis and processing of one or more of said electromyographic signals (S_{EMG})

21. An Apparatus for processing physiological signals and generating a signal indicating an imminent loss of balance of a subject, including:
a plurality of buffers (BEMG) for receiving electromyographic signals (S_{EMG}), arranged to receive and make available a plurality of electromyographic signals (EMG) acquired at a plurality of selected muscles of the subject and representative of a detected muscle activity of said muscles of the subject;
a plurality of buffers for receiving brain signals, arranged to receive and make available a plurality of brain signals of the subject, acquired by means of electroencephalography and representative of a cortical activity of the subject during said muscle activity;
a muscle analysis unit configured to analyse and process the received electromyographic signals and generate at least one indicator of normality/abnormality of a muscle activity pattern for said detected muscle activity;
a cortical analysis unit, configured to process the brain signals received and generate cortical response indicators for a cortical response of the subject to said detected muscle activity, which include one or more generalized cortical response indicators for the cortical response generalized over one or more cortical macro-areas and at least one indicator of lateralization of the cortical response, which indicates a normality/abnormality of the involvement of the left and right cortical sides in the cortical response.
a classifier, configured to receive at its input at least one indicator of normality/abnormality of the detected muscle activity pattern and said cortical response indicators and process them by correlating them so as to generate a signal (Aout) indicating an imminent loss of balance of the subject if it detects at least one anomaly in a generalized cortical response over one or more cortical macro-areas, a non-lateralized anomalous cortical response and a simultaneous abnormality of the muscle activity pattern.

22. The processing apparatus according to claim 21, wherein the muscle analysis unit comprises a digitizer block (21) which, by means of a threshold system, processes each electromyographic signal received so as to derive a respective binary digitized muscle activation signal (OOM,MT) for each electromyographic signal (S_{EMG}) corresponding to a respective monitored muscle, wherein preferably the digitizer block (21) is configured to implement a moving threshold system able to adapt one or more thresholds to variations in muscle tone.

23. The processing apparatus according to claim 22, wherein the muscle analysis unit generates a muscle contraction reference signal MT in response to a contraction of a reference muscle detected by the analysis and processing of one or more of said electromyographic signals (S_{EMG}).

24. The processing apparatus according to claim 22, wherein the muscle analysis unit comprises an MAP extractor block (22) which receives at its input the muscle activation binary signals (OOMx) derived from the electromyographic signals (S_{EMG}) and processes them to extract at least one muscle activity pattern MAP for the detected muscle activity, wherein the MAP is in particular a directional data structure such as a vector.

25. The processing apparatus according to claim 24, wherein the muscle analysis unit comprises a MAP-based scoring block (23), which generates a score (MAScore) indicating a normality/abnormality for each extracted MAP, preferably using a scoring method which quantifies a degree of similarity between the MAP pattern under analysis and a standard muscle behaviour model.

26. The processing apparatus according to claim 25, wherein the muscle analysis unit comprises a threshold decider (24), which receives at its input the MAP normality/abnormality indicating scores (MAScore(k)) and outputs respective binary indicators (MA(k)) of the normality/abnormality of a detected muscle activity pattern.

27. The processing apparatus according to claim 24, wherein the MAP extractor block (22) and preferably the MAP-based scoring block (23) and/or the threshold decider (24) is/are respectively configured to extract an MAP pattern of detected muscle activity, a score indicator (MASCore(k)) and/or a binary indicator (MA(k)) of normality/abnormality of the MAP, for each contraction of a reference muscle.

28. The processing apparatus according to claim 21, further comprising an updating unit (uMAU) for the muscle analysis, configured to receive at its input a plurality of extracted muscle activity patterns MAP and generate or update a standard muscle behaviour model; and/or configured to receive at its input a plurality of previous indicator scores (MAScore(k-x)) of MAP normality/abnormality and calculate an updated threshold for the decider (24) for extracting the binary indicator (MA(k)).

29. The processing apparatus according to claim 21, wherein the cortical analysis unit includes a section (26a) for time-frequency analysis with sliding windows and/or a band multiplexing section (26b) for multiplexing the brain signals in a plurality of frequency bands of interest for processing of the brain signals (S_{EEG}), wherein the bands of interest include one or more, preferably all, of the following frequency bands: θ (4-7 Hz), α (8-12 Hz), β I (13-15 Hz), β II (16-20 Hz), and β III (21-40 Hz).

30. The processing apparatus according to claim 21, wherein the brain analysis unit comprises an extractor block (26) configured to extract a first level cortical response indicator (\hat{m}) for each brain signal (S_{EEG}) and preferably for each band of interest.

31. The processing apparatus according to claim 30, further comprising a generalization section which, based on said one or more first level cortical response indicators (\hat{m}), processes the one or more generalized cortical response indicators, each one representative of the normality/abnormality of a cortical response generalized over a respective cortical macro-area, wherein said cortical macro-areas include, in particular, one or more—preferably all—of the following cortical macro-areas: supplementary motor area, motor area, sensory-motor area and parietal area.

32. The processing apparatus according to claim 30, further comprising a lateralization section which, based on said one or more first level cortical response indicators (\hat{m}), generates one or more cortical response lateralization indicators that provide an indication of an involvement of the left and/or right cortical side in the cortical activity analysed.

33. The processing apparatus according to claim 21, wherein the one or more generalized cortical response indicators and/or the at least one cortical response lateralization indicator generated for the classifier (CL) are binary indicators and/or are generated for each of a plurality of frequency bands of interest.

34. The processing apparatus according to claim 21, wherein the cortical response indicators, the at least one indicator of normality/abnormality of the MAP and/or said signal (Aout) indicating an imminent loss of balance are generated for each contraction of a reference muscle detected by the muscle analysis unit.

35. The processing apparatus according to claim 21, wherein the classifier is a logical classifier with at least three classification levels (CL1;CL2;CL3), in particular comprising a first classifier level (CL1a,CL1b) configured to detect the presence of anomalies in the generalized cortical response over one or more macro-areas, a second classifier level (CL2) configured to detect the presence of abnormal non-lateralized cortical responses and a third classifier level (CL3) configured to detect a simultaneous abnormality of the muscle activation pattern of the selected muscles.

36. (canceled)

37. A detection system for preventive detection of a fall of a subject, comprising:
an acquisition unit comprising a plurality of EMG sensors and a plurality of EEG sensors wearable by the subject and respectively able to acquire, in a continuous and synchronous manner, a plurality of electromyographic signals (EMG) from a plurality of selected muscles of the subject and representative of a detected muscle activity of said muscles of the subject, and a plurality of brain signals (EEG) representative of a cortical activity of the subject during said muscle activity;

21. essing apparatus according to claim 21, connected to said acquisition unit for receiving said plurality of signals.

38. The detection system according to claim 37, further comprising:
a corrective and/or preventive action implementation unit (30), wearable by the subject and connected to the processing apparatus (20), the implementation unit (30) being configured to receive said signal (Aout) indicating an imminent loss of balance and implement at least one corrective action able to prevent falling of the subject and/or at least one preventive action able to limit the effects of an imminent fall of the subject.

39. The detection system according to claim 37, wherein the acquisition unit comprises a plurality of electrodes, in particular at least 15, able to be preferably positioned in the following positions of the 10-20 international system: F3, Fz, F4, C3, Cz, C4, Cp5, Cp1 Cpl, Cp6, P3, Pz, P4, AFz and A2, wherein the AFz electrode is preferably used as a ground electrode and the A2 position electrode as a reference electrode.
